(19) United States
(12) Patent Application Publication
(10) Pub. No.: US 2009/0044815 A1
(43) Pub. Date: Feb. 19, 2009

(54) FOOT AND HEEL SKIN SHIELD SYSTEM

(76) Inventors: Arnold Bruckner, Brooklyn, NY (US); Holly M. Lidowski, West Babylon, NY (US); Leslie Strauss, Dix Hill, NY (US); Karl K. Eilender, New York City, NY (US)

Correspondence Address: LAW OFFICE OF MARC D. MACHTINGER, LTD., 750 W. LAKE COOK ROAD, SUITE 350, BUFFALO GROVE, IL, 60089, US

(21) Appl. No.: 11/839,419

(22) Filed: Aug. 15, 2007

(51) Int. Cl. A61F 13/00; A61F 13/06

(52) U.S. Cl. 128/894; 128/893

(57) ABSTRACT

A foot and heel skin shield system for preventing and treating pressure sores on a person's foot and calf and for managing discharged body fluids from the person's foot and calf is disclosed. The system has two main components, a heel lift and a lubricated disposable calf liner pad. An optional foot stabilizer pad may also be used as part of the system. The system may also include a heel bootie. The lubricated disposable calf liner pad, foot stabilizer pad, and heel boot can have a first lubricant having silicone and a second lubricant having a lubricating, skin protecting, and moisturizing emollient applied to the first lubricant.

### FIELD OF THE INVENTION

The present invention relates to heel lift systems. More specifically, the present invention relates to a heel lift system having a disposable calf liner pad for treating, reducing, or preventing bedsores, pressure sores, decubitus ulcers and similar lesions by pressure, friction, and shearing forces as well as moisture applied to a person's foot and calf while managing fluids discharged from the foot and calf of the person.

### BACKGROUND OF THE INVENTION

It is well established that recent advances in medicine have resulted in longer life expectancies. These changes in life expectancy when coupled with changes in population levels have resulted in a population of elderly people that is much greater than ever before. In the United States, it is an accepted fact that these longer life expectancies carry with them an ever increasing cost of health care. Prevention of untoward medical conditions, such as skin breakdown, can save million of heath care dollars annually.

Skin breakdown, commonly known as bedsores, pressure sores, or decubitus ulcers (hereinafter collectively referred to as bedsores), are ancient problems which recently have begun to reach catastrophic proportions due to the growing population of elderly people. Bedsores are open ulcerations which generally appear in the skin which covers a bony prominence, such as the hip, coccyx, heel, elbow, and head. Ulceration can occur because of compression of tissue between external sources of pressure, friction, and shearing against bony prominences beneath the skin. As the severity of a bedsore increases, the wound may extend to the muscles and bone. These wounds are responsible for causing extensive disabilities, pain, discomfort, and have an associated with them extensive costs of care.

Heel breakdown is the second most common site of skin breakdown. For hospitalized patients, 10 to 18 percent of bedsores are found on the heels. Heel ulcers result when there is a break in the dermal barrier with subsequent erosion of the dermis and underlying subcutaneous tissue.

Bedsores occur in all care settings, including hospitals, rehabilitations centers, long term care facilities, and home care. The vast majority of bedsores can be prevented, yet in the United States conservative estimates indicate the well over two million people in hospitals and nursing care facilities suffer from these lesions. This number does not include those patients receiving care at home. Due to the ever increasing number of patients at risk for, and suffering from, bedsores, and the increased costs associated with the treatment of these sores, the need to prevent bedsores is becoming more acute.

Patients experiencing long hospital stays caused by postoperative complications, chronic disease, or complication injuries are a risk of developing bedsores. Other factors, such as whether a patient is malnourished, incontinent, and or experiencing an altered mental status, can contribute to the rapid development of bedsores. For skin that is delicate, dry, or compromised by any acute or chronic illness, as is common in elderly patients, these factors work together toward the rapid formation of bedsores. For example, the skin of aged people tends to be increasingly sensitive and tends to be less supple, less hydrated, and even more erythematous (abnormally red due to inflammation and capillary congestion) than that of younger people. Studies indicate that bedsores can start to develop in as short a period of time as between one to six hours.

The main causes of bedsores are the forces of pressure, friction, and shearing. Moisture, particularly from bodily fluids, is also an important factor in the formation of bedsores. Together, these forces can cause microcirculatory crimping and tissue deformation. As a result, the underlying blood vessels can be totally or partially occluded, thus depriving the tissue cells of oxygen and other nutrients necessary for cellular viability. Lymphatic drainage can also be affected by these combined forces, resulting in an accumulation of the products of breakdown from the cells' metabolism. When the cells' energy stores are exhausted, the cells begin to fail and normal cellular processes cease. Eventually these cells will die. This progresses to necrosis of the tissue, resulting in the formation of an ulceration and wound.

Pressure causes an external negative force at the interface between the soft tissues of the body and the supporting surface of the bed, or other contact surface, such as the foot pedals of a wheelchair or the surface of the floor. The initial external for of pressure, when combined with friction and shear, is capable of damaging the skin and causing even more severe injuries, including the development of bedsores.

Friction is the resistance to sliding motion of two bodies pressed against one another. The general term friction encompasses static friction and dynamic friction. Static friction results from the resistance to motion in overcoming inertia. Dynamic friction is created by the irregularities of the two surfaces interlocked with one another. A significant force is required to overcome status friction and thus to obtain sliding movement of two bodies with respect to one another Static friction ceases to be a significant factor after sliding motion has been achieved between the two bodies. For instance, when a patient is slipping down against sheets while the patient is sitting up in bed, static friction occurs between the skin and the sheets. As another example, consider a patent being positioned in a bed by sliding across the bed linens. In this example, the patient's skin is subject to dynamic friction between the skin and the linens.

Friction has the effect of causing heat, as well as injury, and may cause the skin to abrade and cause a blister or an open break in the skin's surface. The skin will become reddened. There may also be flaking of the stratum corneum. The tissues will become damaged until the epidermis of the affected tissues ruptures, forming a crater. Friction blisters can easily occur on the foot and ankle areas. Anyone who has suffered from a blister while wearing a new pair of shoes has experienced how painful and sensitive even a small blister can be. Even a tiny break in the skin may allow bacteria to enter and cause a painful wound to form. Feet that tend to perspire more are subject to a higher risk of skin breakdown, and the corresponding development of bedsores, due to the moisture on the skin.

Shearing forces are the internal forces within the skin and supporting tissues that result from the subtle and not so subtle movements of the patient while the patient remains stationary with respect to the bed or wheelchair. For example, when a bedridden patient moves his or her arms or twists his or her shoulders, but their hips remain stationary, specifically, the skin on the patient's buttocks remains stationary on the bed, shear forces are created in the skin tissue and supporting tissues. As a result of these shearing forces, capillaries become damaged, distorted, crimped and occluded. This leads to ischemia (deprivation of blood supply to an area) of tissue, which can lead to an area of dead skin, which can develop into a bedsore. Additionally, when friction and shearing occur there is also a build up of heat, which is caused by the rubbing irritation of the skin's surface. The increased heat can lead to microscopic openings in the skin surface, which allows the skin to be susceptible to infection or other irritants.

Furthermore, the introduction of liquid, such as from perspiration or leakage of incontinence from an incontinent patient, can accelerate the formation of bedsores. The presence of liquid softens the skin and leads to the separation of the layers of the skin. These factors contribute to the formation of skin breakdown, resulting in wound formation.

In U.S. Pat. No. 4,572,174, which issued Feb. 25, 1986, we disclosed a bed pad structure intended to relieve the pressure, friction, and shear forces which are now recognized as principal causes of bedsores, pressure sores and decubitus ulcers in bedridden and chair ridden patients. We disclosed a low friction bed pad structure having a pouch portion between a woven fabric upper porous sheet and a lower flexible nonporous sheet. The pouch portion permitted insertion and removal of a lubricated sheet which exuded lubricant that seeped through the pores in the upper sheet to a patient's body when it applied pressure to the pad being held stationary on a bed, to reduce friction between the patient's body and the bed or other supporting surface to which the pad was attached.

Some of the shortcomings of the pads disclosed in U.S. Pat. No. 4,572,174 were addressed in U.S. Pat. No. 4,959,059, which issued Sep. 23, 1990. In U.S. Pat. No. 4,959,059, we disclosed a multilayer low friction ambulatory pad for treating or preventing bedsores and pressure sores and for managing fluids discharged from a person's body. The pad has a first slippery nonporous layer on which is a moisture absorbent second layer. A slippery, thin, porous third layer is on the second layer. The three layers are peripherally bonded to form a continuous seam which permits unbonded areas of the layers to slide slightly with respect to each other. The third layer may be sprayed with a filmy, dry, slippery fourth layer. A fifth layer of lubricating material, which may be a microencapsulated lubricant or a free lubricant, is applied to the third and fourth layers to form a very slippery top surface. The fourth and fifth layers do not clog the pores of the third layer, thus allowing passage of air and fluids to the absorbent second layer. In some variants of the pad, the absorbent layer is omitted. The pad can be discarded after a single use.

While the above referenced U.S. patents focused on bed pads, the bed pad technology can be improved upon and used to prevent pressure wounds on other parts of the body. These other areas include the foot, heel, and calf. As mentioned above, these areas of the body are also susceptible to pressure wounds.

The same “at risk” population of patients mentioned above is also susceptible to the onset of contractures. Patients with impaired mobility may develop a contracture when a patient if immobilized for a long period of time. In these situations, the patient is unable to maintain the normal extension of their legs. When a leg is bent for extended periods of time, the circulation becomes impaired, arteries and veins can become crimped and blood flow is diminished. If a leg is allowed to remain in a bent position, tendons, muscles, and ligaments may become contracted and shortened, resulting in the formation of a leg contracture. Contractures may occur even when there are no arthritic changes. Patients that develop contractures typically require extensive physiotherapy and other rehabilitative therapies. In treating contractures, which are very difficult to treat, early start of physiotherapy is crucial. However, physiotherapy is performed on a special schedule. As a result of this scheduling, the patient often spends hours at a time with the leg stationary and flexed. These idle periods counteract the results of the physiotherapy and other rehab measures.

Another problem encountered by bed ridden patients is the development of contractures of the feet and lower leg. Unsupported feet and lower extremities may develop flexion and atrophy of the muscles. This can result in the patient not being able to maintain normal body alignment or being able to flex their feet to an upward position, a condition referred to as “foot drop” or foot destabilization. In patients suffering foot destabilization the foot flexes inward and downwards.

Treatment for, or for the prevention of, foot destabilization often involves maintaining the foot in the flexed position. However, as the foot may flex downward, pressure develops between the foot and the device maintaining the foot in the flexed position. This pressure can lead to further occurrence of bedsores. Thus, it would be desirable for a device used to treat or prevent foot drop to treat or prevent the development of pressure wounds at the same time.

There remains a need for an improved foot and heel skin shield system. Specifically, there remains a need for an improved foot and heel skin shield system for reducing or preventing bedsores caused by pressure and friction applied to a person's foot and calf while managing fluids discharged from the foot and calf of the person. Furthermore, there remains a need for an improved foot and heel skin shield system for reducing or preventing bedsores caused by pressure and friction applied to a person's foot during treatment for, or for the prevention of, foot drop.

### SUMMARY

In view of the deficiencies described above, it is an object of the present invention to provide an improved foot and heel skin shield system. Specifically, it is an object of the present invention to provide an improved foot and heel skin shield system for reducing or preventing bedsores caused by pressure and friction applied to a person's foot and calf while managing fluids discharged from the foot and calf of the person.

It is a further object of the present invention to provide an improved foot and heel skin shield system for the treatment of prevention of foot drop in bed ridden patients.

The present invention is a foot and heel skin shield system for preventing and treating pressure sores on a person's foot and calf and for managing discharged body fluids from the person's foot and calf. The foot and heel skin shield system has two main components, a heel lift and a lubricated disposable calf liner pad. An optional foot stabilizer pad may also be used as part of the system.

The heel lift has a contoured bottom support for receiving a person's foot and calf thereon and two side supports. The lubricated disposable calf liner pad is placed between the contoured bottom support and the person's foot and calf. Preferably the lubricated disposable calf liner pad has at least one lubricant applied thereto.

In various preferred embodiments, the lubricated disposable calf liner pad can be double lubricated. A first lubricant can include silicone. The first lubricant serves to create an almost friction-free film between the foam rubber of the lubricated disposable calf liner pad and a second lubricant, discussed below. The second lubricant can comprise a lubricating, skin protecting, and moisturizing emollient applied to the first lubricant. In various preferred embodiments, the first lubricant and the second lubricant are applied to the foam rubber simultaneously using a spray system, such as a swirl or “tornado” inducing spray system, which results in the emollient of the second lubricant being microencapsulated within silicone beads of the first lubricant.

In various preferred embodiments, the present invention can also include a foot stabilizer pad. In various preferred embodiments, the foot stabilizer pad can be treated with the first lubricant and second lubricant as discussed above. Hook and loop closure tabs can be used to secure the foot stabilizer pad to the side supports of the heal lift.

In other various embodiments, the present invention can also include a heel bootie. In various preferred embodiments, the heel bootie can be treated with the first lubricant and second lubricant as discussed above. Hook and loop tabs can be used to secure the heel bootie around a patient's ankle.

Other features and advantages of the invention will be apparent from the following detailed description taken in conjunction with the following figures, wherein like reference numerals represent like features.

### BRIEF DESCRIPTION OF THE DRAWINGS

FIG. 1 is a perspective view of heel and skin shield system according to the present invention.

FIG. 2 is a perspective view of a heel and skin shield system according to the present invention in use.

FIG. 3 is a perspective view of a heel and skin shield system according to the present invention in use with an optional foot stabilizer pad.

FIG. 4 shows a disposable calf liner pad according to an aspect of the present invention.

FIG. 5 shows the optional foot stabilizer pad according to the present invention.

FIG. 6 shows an optional heel bootie according to an aspect of the present invention.

### DETAILED DESCRIPTION OF THE INVENTION

While this invention is susceptible of embodiments in many different forms, there are shown in the drawings and will herein be described in detail, preferred embodiments of the invention with the understanding that the present disclosure is to be considered as an exemplification of the principles of the invention and is not intended to limit the broad aspect of the invention to the embodiments illustrated.

The present invention is a foot and heel skin shield system for preventing and treating pressure sores on a person's foot and calf and for managing discharged body fluids from the person's foot and calf. The present invention protects and supports the lower leg and foot, helping to maintain normal body and joint alignment. The present invention also protects against the formation of contractures occurring at the ankle and foot. Additionally, the present invention protects against skin breakdown, protecting the skin of the calf and foot.

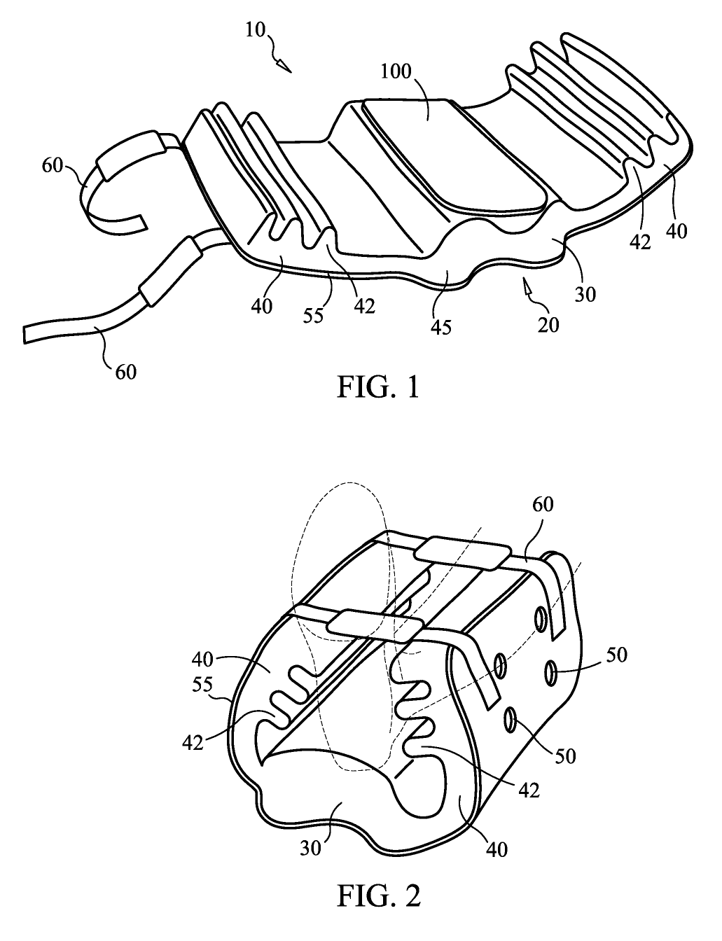

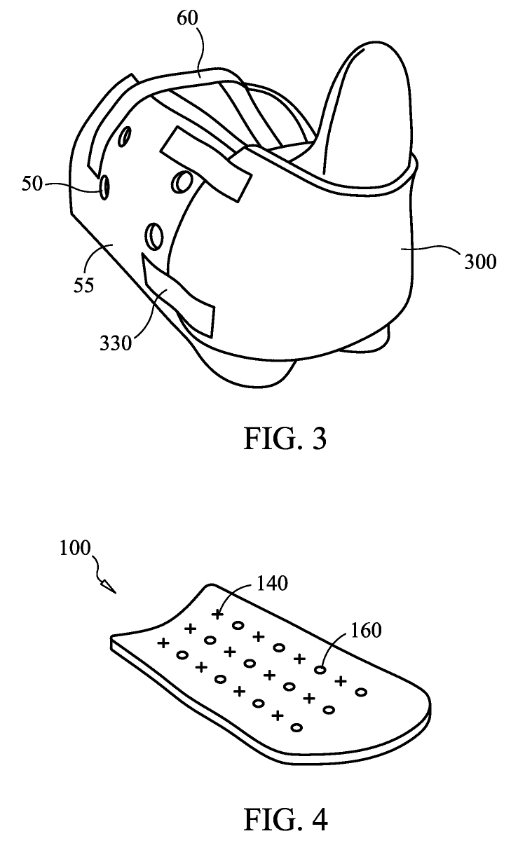

FIG. 1 is a perspective view of heel and skin shield system according to the present invention. FIG. 2 is a perspective view of a heel and skin shield system according to the present invention in use. The foot and heel skin shield system 10 has two main components, heel lift 20 and a lubricated disposable calf liner pad 100. An optional foot stabilizer pad 300 may also be used as part of the system 10. FIG. 3. is a perspective view of a heel and skin shield system according to the present invention in use with the optional foot stabilizer pad. The present invention helps stabilize the foot above the surface of the bed, preventing the foot from coming in contact with the bed surface. Thus, the foot is not subjected to pressure forces since the foot and ankle are lifted off the bed surface.

The heel lift 20 has a contoured bottom support 30 for receiving a person's foot and calf thereon and two side supports 40. The bottom support 30 acts to prevent the heel from touching any obstructions that may cause or prevent the healing of bedsores on the heel. In various preferred embodiments, the contoured bottom support 30 and the side supports 40 are constructed from ventilated foam material 45, such as that manufactured by Crest Foam Industries. In a preferred embodiment, the contoured bottom support 30 is about 2¾ inches high and about 12 inches in length.

Preferably the ventilated foam material has a plurality of passages 50 there through to allow the free movement of air about the foot and calf, thus allowing the skin to “breathe” and minimize the capture of perspiration. In a preferred embodiment, there are ten passages 50, each about ¾ inches in diameter.

As shown, the side supports 40 and the contoured bottom support 30 are constructed from a single piece of ventilated foam material 45. The side supports 40 may also have a plurality of crests 42 formed therein, these crests 42 serve to support and stabilize the foot and calf as well as promote air circulation about the limb. In a preferred embodiment, the crests 42 have a total height of about 2 inches.

A backing material 55 may also be applied to the ventilated foam material 45. In a preferred embodiment, the backing material can include a spun bond polypropylene flame laminated to unifoam S 82 S which is flame laminated to a polyester polypropylene tricot outer fabric. Hook and loop closure tabs 60, or other means known in the art, can be used to secure the person's foot and calf between the side supports 40 and on top of the contoured bottom support 30.

The lubricated disposable calf liner pad 100 is placed between the contoured bottom support 30 and the person's foot and calf. The lubricated disposable calf liner pad 100 protects against the skin against friction and shear forces that may cause skin breakdown. FIG. 4 shows a disposable calf liner pad according to an aspect of the present invention. In various preferred embodiments, the disposable calf liner pad 100 is constructed of a tricot fabric flame laminated to unifoam S 82 S which is flame laminated to a non-woven spun bond. Preferably the lubricated disposable calf liner pad 100 has at least one lubricant applied thereto, such as to the non-woven spun bond.

In various preferred embodiments, the lubricated disposable calf liner pad 100 is double lubricated. A first lubricant 140 can include silicone. For example, the first lubricant 140 can made from Emulsion 365 from Dow Corning and Akorex L. The first lubricant 140 serves to create an almost friction-free film between the foam rubber 120 and second lubricant 160, discussed below. The first lubricant 140 is applied in an amount effective to reduce friction. This effective amount of the first lubricant 140 is in the range of about 0.1 grams per square into to about 1.0 grams per square inch.

The second lubricant 160 can comprise a lubricating, skin protecting, and moisturizing emollient applied to the first lubricant 140. The emollient of the second lubricant 160 is applied in an amount effective to treat and reduce the possibilities of a patient developing bed sores. This effective amount for the second lubricant 160 is in the range of about 0.1 grams per square into to about 1.0 grams per square inch.

In various preferred embodiments, the first lubricant 140 and the second lubricant 160 are applied to the disposable calf liner pad 100 simultaneously using a spray system, such as a swirl or “tornado” inducing spray system, which results in the emollient of the second lubricant 160 being microencapsulated within silicone beads of the first lubricant 140. The results of this microencapsulating process means that the emollient of the second lubricant 160 is not exposed while disposable calf liner pad 100 is stored. The weight of a patient being placed on the disposable calf liner pad 100 releases the emollient of the second lubricant 160 from the silicone beads of the first lubricant 140.

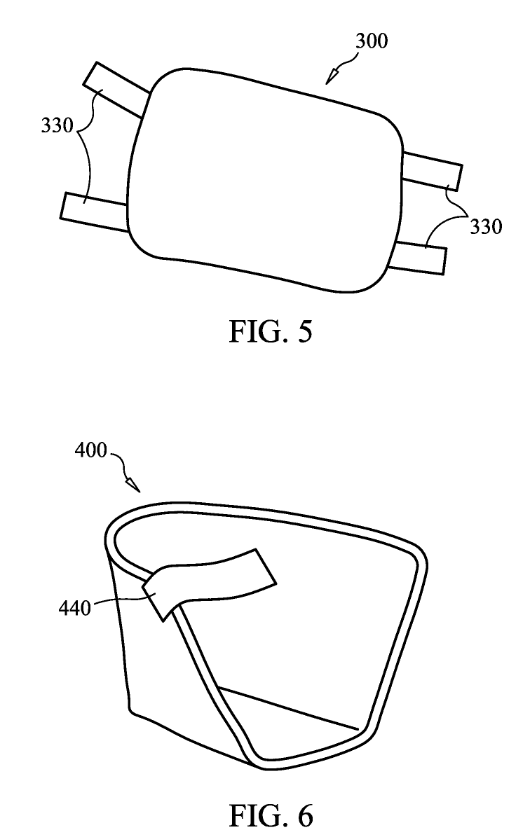

In various preferred embodiments, the present invention can also include a foot stabilizer pad 300. FIG. 3. is a perspective view of a heel and skin shield system according to the present invention in use with an optional foot stabilizer pad. FIG. 5 shows the optional foot stabilizer pad according to the present invention. In various preferred embodiments, the foot stabilizer pad 300 is constructed of a tricot fabric flame laminated to unifoam S 82 S which is flame laminated to a non-woven spun bond. Preferably the foot stabilizer pad 300 has at least one lubricant applied thereto, such as to the non-woven spun bond. Hook and loop closure tabs 330 can be used to secure the foot stabilizer pad 300 to the side supports 40 of the heal lift 20.

In other various embodiments, the present invention can also include a heel bootie 400. FIG. 6 shows an optional heel bootie according to an aspect of the present invention. The heel bootie 400 can be constructed from the same foam rubber type material as the disposable calf liner pad 100. Likewise, in various preferred embodiments, the heel bootie 400 can be treated with the first lubricant 140 and second lubricant 160 as discussed above. Hook and loop tabs 440 can be used to secure the heel bootie 400 around a patient's ankle. The heel bootie 400 may also be suitable for treating, reducing, or preventing bed sores located on other areas of the body, including, for example, the elbow.

While specific embodiments have been illustrated and described, numerous modifications come to mind without significantly departing from the spirit of the invention and the scope of protection is limited by the scope of the accompanying claims.

What is claimed is:

1. A foot and heel skin shield system for preventing and treating pressure sores on a person's foot and calf comprising:
a heel lift comprising:
a contoured bottom support for receiving said person's foot and calf thereon,
two side supports, and
means for securing said person's foot and calf between said side supports and on top of said contoured bottom support, and
a lubricated disposable calf liner pad.

2. The heel lift system for preventing and treating pressure sores on a person's foot and calf according to claim 1 wherein said lubricated disposable calf liner pad comprises a first lubricant comprising silicone.

3. The foot and heel skin shield system for preventing and treating pressure sores on a person's foot and calf according to claim 2 wherein said first lubricant comprises Dow Corning 365 NF Emulsion.

4. The foot and heel skin shield system for preventing and treating pressure sores on a person's foot and calf according to claim 2 wherein said lubricated disposable calf liner pad further comprises a second lubricant.

5. The foot and heel skin shield system for preventing and treating pressure sores on a person's foot and calf to claim 4, said first lubricant and said second lubricant being applied to said lubricated disposable calf liner pad such that said second lubricant is microencapsulated in said first lubricant.

6. The foot and heel skin shield system for preventing and treating pressure sores on a person's foot and calf according to claim 4, wherein said first lubricant comprises an effective amount to reduce friction between foam rubber of said lubricated disposable calf liner pad and said second lubricant.

7. The foot and heel skin shield system for preventing and treating pressure sores on a person's foot and calf according to claim 6, wherein said effective amount is in the range of about 0.1 grams per square inch to about 1.0 grams per square inch.

8. The foot and heel skin shield system for preventing and treating pressure sores on a person's foot and calf according to claim 4, wherein said second lubricant comprises an effective amount of emollient in the range of about 0.1 grams per square inch to about 1.0 grams per square inch.

9. The foot and heel skin shield system for preventing and treating pressure sores on a person's foot and calf according to claim 1 further comprising a disposable foot stabilizer pad supporting the sole of said person's foot and having means for attaching to said side supports.

10. The foot and heel skin shield system for preventing and treating pressure sores on a person's foot and calf according to claim 9, wherein said disposable foot stabilizer pad comprises a first lubricant and a second lubricant.

11. The foot and heel skin shield system for preventing and treating pressure sores on a person's foot and calf according to claim 1, wherein said side supports comprise ventilated foam having a plurality of crests formed therein and a plurality of passages therethrough.

12. The foot and heel skin shield system for preventing and treating pressure sores on a person's foot and calf according to claim 11, wherein a single piece of said ventilated foam forms said contoured bottom support and said side supports.

13. The foot and heel skin shield system for preventing and treating pressure sores on a person's foot and calf according to claim 1, wherein said means for securing said person's foot and calf between said side supports and on top of said contoured bottom support comprises at least one hook and loop closures tab for selectively connecting said side supports.

14. The foot and heel skin shield system for preventing and treating pressure sores on a person's foot and calf according to claim 9, wherein said means for attaching said disposable foot stabilizer pad to said side supports comprises a plurality of hook and loop closure tabs.

15. The foot and heel skin shield system for preventing and treating pressure sores on a person's foot and calf according to claim 1, further comprising a lubricated heel bootie.

16. The foot and heel skin shield system for preventing and treating pressure sores on a person's foot and calf according to claim 15, wherein said lubricated heel bootie comprises a first lubricant and a second lubricant.

17. The foot and heel skin shield system for preventing and treating pressure sores on a person's foot and calf according to claim 15, wherein said lubricated heel bootie comprises hook and loop tabs.
